(19) United States
(12) Patent Application Publication
(10) Pub. No.: US 2023/0138286 A1
(43) Pub. Date: May 4, 2023

(54) METAL-MODIFIED NANOPARTICLE ENABLED DENTAL RESINS FOR PREVENTION OF DENTAL CARIES

(71) Applicant: KISMET TECHNOLOGIES LLC, Winter Park, FL (US)

(72) Inventor: Christina H. Drake, Winter Park, FL (US)

(21) Appl. No.: 17/973,720

(22) Filed: Oct. 26, 2022

Related U.S. Application Data
(60) Provisional application No. 63/272,924, filed on Oct. 28, 2021.
(60) Provisional application No. 63/272,930, filed on Oct. 28, 2021.

(51) Int. Cl. A61K 6/889; A61C 13/15; A61K 6/70; A61K 6/62; A61K 6/76

(52) U.S. Cl. CPC A61K 6/889; A61C 19/003; A61K 6/62; A61K 6/70; A61K 6/76

(57) ABSTRACT

A dental resin composition that includes a dental resin. The composition includes metal-modified cerium oxide nanoparticles (mCNPs) having a predominantly 3+ cerium surface charge and in a range of about 3-25 nm in size and mixed in an amount that is in a range of about 0.01 to 0.1 weight percentage of a mixture having the dental resin and the mCNPs. The m is an antimicrobial promoting metal that is non-ionizing. The mCNPs effectuate release directed hydrogen peroxide (H₂O₂) in an oral cavity that is then used against bacteria in the oral cavity to prevent local acidification of a tooth on which a therapeutically effect amount of the mixture is applied and cured. The dental resin composition includes one of an acrylic-based polymer resin, a methyl methacrylate-based resin, photopolimerisable resin composite, and urethane dimethacrylate-based dental resin.

### CROSS-REFERENCE TO RELATED APPLICATIONS

This application claims priority benefit of U.S. Provisional Application No. 63/272,924, titled “METAL-MODIFIED NANOPARTICLE ENABLED DENTAL RESINS FOR PREVENTION OF DENTAL CARIES,” filed Oct. 28, 2021, which is incorporated herein by reference in its entirety. This application also claims priority benefit of U.S. Provisional Application No. 63/272,930, titled “NANOPARTICLES TO PROMOTE WOUND HEALING AND ANTIMICROBIAL INFECTION CONTROL,” filed Oct. 28, 2021, incorporated herein by reference in its entirety.

### BACKGROUND

Embodiments relate to the field of medical science and, more specifically, the field of dentistry to prevent dental caries using a dental resin composite with metal-modified nanoparticles.

For as long as humans have existed, tooth decay is an on-going issue. For example, in the United States, 94% of adults will have experienced dental caries, known as tooth decay. Most of these occur during a person’s childhood and is a result of the growth of bacteria and/or biofilms that cause the local pH to drop on a person’s tooth and demineralization of the tooth, which results in tooth decay.

Dental caries is amongst one of the most preventable childhood diseases, as it commonly afflicts younger people. Brushing a person’s teeth is not sufficient for most to prevent cavities.

### SUMMARY

Embodiments relate to the field of medical science and, more specifically, the field of dentistry to prevent dental caries using a dental resin composite with metal-modified nanoparticles.

An aspect of the embodiments includes a dental resin composition that includes a dental resin. The composition includes metal-modified cerium oxide nanoparticles (mCNPs) having a predominantly 3+ cerium surface charge and in a range of about 3-25 nm in size and mixed in an amount that is in a range of about 0.01 to 0.1 weight percentage of a mixture having the dental resin and the mCNPs, where m is an antimicrobial promoting metal that is non-ionizing. The mCNPs effectuate release directed hydrogen peroxide (H₂O₂) in an oral cavity that is then used against bacteria in the oral cavity to prevent local acidification of a tooth on which a therapeutically effect amount of the mixture is applied and cured.

An aspect of the embodiments includes a method of treating teeth that includes forming the resin composition on enamel of each tooth to be treated. The method includes promoting by the resin composition remineralization of tooth material to further strengthen teeth against decay.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1A illustrates a subject tooth in a clean state;

FIG. 1B illustrates a subject tooth of FIG. 1A with a coating of an oxidizing nanoparticle resin composite coated on the tooth;

FIG. 2 illustrates a coated subject tooth of FIG. 1B in the mouth with bacteria;

FIG. 3 illustrates a coated subject tooth of FIG. 2 releasing directed hydrogen peroxide to degrade or destroy caries-causing bacteria;

FIG. 4 illustrates the coated subject tooth of FIG. 3 with the bacteria degraded or destroyed;

FIG. 5 illustrates a graphical representation of an efficacy test of AgCNP2 against Streptococcus mutans;

FIG. 6A illustrates a photograph of a control surface in a pediatric facility that was not cleaned, and after 28 days P. aeruginosa, K. pneumoniae and S. aureus are present on the surface;

FIG. 6B illustrates a photograph of an AgCNP2 with a binder treated surface (<0.1 weight %) (“NanoRAD”) and the untreated surface, where neither were cleaned for 28 days, where the NanoRAD treated surface prevented bacteria survival for 28 days, and the untreated surface matched the control surface;

FIG. 7 illustrates a graphical representation of efficacy tests of AgCNP2 (NanoRAD) at different incubation times of 10, 30, 60 and 120 minutes with Escherichia coli;

FIG. 8 illustrates a graphical representation of efficacy tests of AgCNP2 (NanoRAD) at different incubation times of 10, 30, 60 and 120 minutes with Staphylococcus epidermidis;

FIG. 9A illustrates a photograph of S. epidermidis dried on glass and treated with a positive control medium in three separate areas/sections 1, 2 and 3;

FIG. 9B illustrates a table of results over two hours of the S. epidermidis dried on the glass in areas/sections 1, 2 and 3;

FIG. 10A illustrates a photograph of S. epidermidis dried on glass coated with AgCNP2 and polyurethane in three separate areas/sections 1, 2 and 3; and

FIG. 10B illustrates a table of results over two hours of the S. epidermidis dried on the glass coated with AgCNP2 and polyurethane in three separate areas/sections 1, 2, and 3.

### DETAILED DESCRIPTION

The inventor has surprisingly determined that metal-modified cerium oxide nanoparticles (mCNPs), as described herein, destroys caries-causing bacteria that is found in the mouth, with an elicit response to the caries-causing bacteria by the release of hydrogen peroxide (H₂O₂) from a dental resin composite including the metal-modified nanoceria described herein below. The inventor has determined that the metal-modified nanoceria of about 0.01 to 0.1 weight (wt.) % in a dental resin composite is non-toxic.

The inventor has determined that most bio-safe antimicrobials and antiseptics are oxidizing and not able to switch their response based on the type of cell presented to it. Nanoceria, known as an antioxidant, has had less success as an antimicrobial. This is because the rate of the surface reaction on nanoceria is low compared to the number of species it would need to deactivate in a given time period ((i.e., 10⁵ viral load or 10⁸ colony-forming unit (CFU)) of bacteria). The mCNPs, described herein, allows for quicker electron recovery of the nanoceria so that it can quickly regenerate its surface creation of oxidizing species. When water is present, this reaction can occur rapidly by digestion of the water by mCNPs, described herein, to these oxidizing species. When a healthy cell is present, the local environment (i.e., potential hydrogen (pH)) switches the behavior of the mCNPs to be free radical scavenging instead.

The inventor has determined that the mCNPs, as described herein, can be applied to many forms of dental appropriate resins, such as acryl-based polymers, to prevent biofilm and bacteria growth on the enamel of the tooth that leads to eventual decay. The inventor has surprisingly discovered that the nanoparticles do not ionize or release agents to the teeth to perform its antimicrobial activity. Instead, it locally creates surface oxidizing reactions when presented with bacteria or viruses, killing them, and preventing further proliferation.

The inventor has determined that the mCNPs, as described herein, can be applied to many forms of dental appropriate resins, such as acryl-based polymers, to assist with a body’s naturally occurring processes to remineralize tooth material to further strengthen teeth against decay and sensitivity.

The mCNPs, described herein, possess Super Oxide Dismutase (SOD) activity, common to many bio-safe forms of nanoceria. Unlike other nanoceria, the “switch -over” reactions on the nanoceria surface are made fast and more potent by the small presence of discrete silver on the surface of the nanoceria. This allows for quick and facile change in surface behavior of the nanoceria between creation of oxidizing species that are harmful to viruses and bacteria, and to free radical scavenging behavior (antioxidant behavior) that is beneficial to healthy cells. This allows for targeted control of surface reactions to bacteria and biofilms on a tooth’s surface without release of agents (ions or other chemicals) that can be irritating or toxic. The antimicrobial effect would persist for the life of the sealant, unlike other current approaches that leach or ionize species to perform antimicrobial functions on the tooth.

The inventor has discovered that the mCNPs, as described herein, are engineered enzymatic-like nanoparticles that can be deployed to teeth in a number of different host resin systems appropriate to dental care such as, without limitation, acryl polymers. Once delivered to the tooth, the nanoparticle resin would be able to kill bacteria and biofilms on the tooth’s surface, preventing local acidification that leads to demineralization of the tooth and eventual tooth decay, or cavities.

### Definitions

Unless specifically stated or obvious from context, as used herein, the term “about” is understood as within a range of normal tolerance in the art, for example, within 2 standard deviations of the mean. “About” can be understood as within 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “composition” or “composite” refers to a product that includes ingredients such as one or more of chemical elements, excipient, diluent, binder, dental resin, or constituent in specified amounts, in addition to any product which results, whether directly or indirectly, from a combination of the ingredients in the specified amounts.

The term “pharmaceutically acceptable” component as used herein refers to an ingredient that is suitable for use with humans and/or animals without undue adverse side effects (such as toxicity, irritation, and allergic response) commensurate with a reasonable benefit/risk ratio.

The term “prevention” or “preventing” of a disorder, disease, or condition as used herein refers to, in a statistical sample, a measurable or observable reduction in the occurrence of the disorder, disease or condition in the treated sample set being treated relative to an untreated control sample set or delays the onset of one or more symptoms of the disorder, disease or condition relative to the untreated control sample set.

As used herein, the terms “subject,” “individual,” or “patient” refers to a human, a mammal, or an animal.

The term “therapeutically effective amount” as used herein refers to an amount of active compound or pharmaceutical agent that elicits the biological or medicinal response in a tissue, system, animal, or human that is being sought by a researcher, veterinarian, medical doctor, or other clinician. A therapeutically effective amount can be given in one or more administrations. The amount of a compound which constitutes a therapeutically effective amount will vary depending on the compound, the disorder and its severity, and the general health, age, sex, body weight and tolerance to drugs of the subject to be treated, but can be determined routinely by one of ordinary skill in the art.

The term “treating” or “treatment” as used herein covers the treatment of a disorder, disease or condition described herein, in a subject, and includes: (i) inhibiting development of a disorder, disease or condition; (ii) slowing progression of the disorder, disease or condition; (iii) inhibiting, relieving, or slowing progression of one or more symptoms of the disorder, disease or condition; and (iv) assisting with a body’s naturally occurring processes to remineralize tooth material to further strengthen teeth against decay and sensitivity.

The term “metal-modified cerium oxide nanoparticles,” “metal-modified ceria nanoparticles,” or “mCNPs” as used herein refers to cerium oxide nanoparticles coated with or otherwise bound to an antimicrobial promoting metal (m) such as silver, gold, copper, platinum, nickel, zinc, iron, titanium, ruthenium, vanadium, and the like. The term “mCNPs” includes AgCNP2, as described herein. In an embodiment, the metal-associated cerium oxide nanoparticles comprise a particle size in the range of 3 nm - 25 nm or from 1 nm - 7 nm. In some embodiments, the mCNP ingredient with predominately Ce 3+ cerium oxide surface charge may have a particle size in the range of 3 nm - 35 nm.

As sometimes used herein, cerium oxide nanoparticles is referred to as “nanoceria.” The mCNP ingredient described herein is a Rapid and Residual Acting Disinfectant (RAD) with nanoparticles, hereinafter referred to as “NanoRAD.”

The inventor has determined that AgCNP1 is catalase mimetic.

The AgCNP1 and AgCNP2 are enzyme mimetic non-stoichiometric nano-cerium oxide. The silver of the synthesis for AgCNP1 and AgCNP2 is a stable metallic silver that is non-ionizing. While not wishing to be bound by theory, the synthesis makes it so that a few atomic layers of the stable metallic silver is anchored into the cerium oxide and contributes to cerium oxide activity (+3 and super oxide dismutase), as opposed to be the direct antimicrobial.

The term “predominant 4+ surface charge” refers to the concentration of cerium ions on the surface and means that the [Ce3+]:[Ce4+] ratio on the surface of the ceriumoxide nanoparticle is less than 50%. In a specific example, cerium oxide nanoparticles having a predominant 4+ surface charge have a [Ce3+]:[Ce4+] ratio that is 40% or less. The term “predominant 3+ surface charge” means that the [Ce3+]:[Ce4+] ratio on the surface of the cerium oxide nanoparticle is greater than 50%.

### Overview

Dental caries occur when bacteria ferments carbohydrates with an acid by-product on the tooth. This leads to acid diffusion into the tooth crystal, eventually leading to diffusion of calcium, phosphate, and carbonate out of the tooth, causing demineralization of the tooth. When this happens, cavities form from the loss of dental material if sufficient remineralization does not occur from the body’s natural processes.

Silver and chlorhexidine are common antimicrobials used as antimicrobials, but suffer from longevity of the solution and potential toxicity and irritation. Fluoride has been used to help support tooth remineralization, but despite water fluorination across the US, most people will still experience dental caries and cavities.

Most dental techniques for reduction of dental caries or prevention of cavities require release of an ion or chemical that is potentially ingested and can cause irritation or other harmful health effects. [See, Cheng et al., “Developing a New Generation of Antimicrobial and Bioactive Dental Resins,” Journal of Dental Research 2017, Vol. 96(8) 855-863, DOI 10.177/0022034517709739., incorporated by reference.]

### Metal-Modified Cerium Oxide Nanoparticle (AgCNP2)

Using a forced hydrolysis reaction, a solution containing silver-modified nanoceria and silver secondary phases were formed, hereinafter referred to as “material.” The material was washed with distilled water. Then, the washed material was treated with ammonium hydroxide (NH₄OH). The material was also treated with a phase transfer complex: mediating aqueous dispersion of dissolved silver, (Ag[(NH₃)₂OH]_{aq}). After treatment, the treated material was washed again, such as by distilled water. In another synthesis that yields silver modified nanoceria, silver nitrate (AgNO₃) and cerium (Ce) are dissolved to form a mixture. Then the mixture is dissolved by hydrogen peroxide (H₂O₂) which causes selective oxidation of Ce³⁺ over silver and the evolution of metallic silver phases on the ceria surface. The formula properties for AgCNP2 is shown below in Table 1.

TABLE 1

| | AgCNP2 | Inorganic Crystal Structure Database No. (ICSD #) |
|---|---|---|
| Ce^{3+:}Ce⁴⁺ (%Ce³⁺)(%) | 53.7 | |
| [Ag]/Ag+Ce] by XPS(%) | 4.6 | |
| SOD Activity (% Inhibition) | 99.2 | |
| Hydrodynamic Diameter (nm) | 31.6 ± 2.4 | |
| Zeta Potential (mV) | 24.1 ± 1.3 | |
| ICPMS Ce concentration (ppb) | 299.2 ±1.3 | |
| E_{corr} (mV) | 217.374 | |
| Metallic Ag | | 44387 |
| CeO₂ | | 55284 |

A process for metal-mediated nanoscale cerium oxide is described in Craig J. Neal et al., titled “schemMetal-Mediated Nanoscale Cerium Oxide Inactivates Human Coronavirus and Rhinovirus by Surface Disruption,” ACS Publications, ACSJCA Aug. 23, 2021, doi.org/10.1021/acsnano.lc04142, incorporated herein by reference in its entirety.

A Zeta-sizer nano was used from Malvern Instruments to determine hydrodynamic diameters and zeta potentials. Tafel analysis for AgCNP2 shows distinct corrosion potentials. E_{corr} values are substantially more noble than pure silver.

A more detailed description of the process for forming AgCNP2 will now be described. First, about 109 mg of cerium nitrate hexa-hydrate (99.999% purity) is dissolved in about 47.75 mL dH₂O in a 50 ml square glass bottom. Then, about 250 µL of 0.2 M aq. AgNO₃ (99% purity) is added to the cerium solution above with the solution vortexed for 2 minutes: Machine: Vortexer. Then, about 2 mL of 3% hydrogen peroxide (stock) is added quickly to the above solution followed by immediate vortexing for 2 minutes at highest rotation speed (in vortexer machine). This solution is stored in dark condition at room temperature with the bottle (50 mL square bottom glass) cap loose to allow for release of evolved gases; solutions are left to age in these conditions for up to 3 weeks (monitoring solution color change from yellow to clear) to create 50 ml total volume of the solution. Particles are then dialyzed against 2 liters of dH2O over 2 days, (dialysis Tubing) with the water changed every 2 hours and stored in the same conditions as for ageing.

The two unique formulations of cerium oxide nanoparticles are produced with surfaces modified by silver nanophases. Materials characterization shows that the silver components in each formulation are unique from each other and decorate the ceria surface as many small nanocrystals (AgCNP1) or as a Janus-type two-phase construct (AgCNP2). The average diameter of AgCNP1 is about 20 to 24 nm, and the average diameter of AgCNP2 is about 3 to 25 nm. However, the inventor prefers the use of AgCNP2, for the reasons stated below.

Each synthesis further possesses unique mixed valency with AgCNP2 possessing a significantly greater fraction of Ce³⁺ states relative to Ce⁴⁺ over AgCNP1. The distinct valence characters, along with incorporation of chemically active silver phases, lead to high catalytic activities for each formulation. AgCNP2 possesses high superoxide dismutase activity, while AgCNP1 possesses both catalase and superoxide dismutase-like enzyme-mimetic activities, ascribed to the catalase activity of ceria and the superoxide dismutase activity from silver phases.

Further, analysis demonstrates that silver incorporated in each formulation is substantially more stable to redox-mediated degradation than pure silver phases: promoting an increased lifetime in catalytic applications and low probability of ionization of the silver phase.

Use of AgCNP2 formulation in effecting antimicrobial properties showed specific activity in tests associated with bacteria, with, among bacteria species tested, AgCNP2 showing substantial activity towards staphylococcus mutants, such as staphylococcus aureus. Staphylococcus mutants include a family of bacteria found in the mouth or oral cavity.

Although the amount is not intended to be limiting, when used in methods of the invention, some preferred amounts of silver percentages associated with the AgCNPs are about 8% to 15% or less.

In other embodiments, disclosed is a method of producing mCNPs, as described herein, may include the metal of silver. Further the AgCNP2 is produced via a method comprising dissolving cerium and silver precursor salts such as cerium and silver nitrates; oxidizing the dissolved cerium and silver precursor salts via admixture with peroxide; and precipitating nanoparticles by subjecting the admixture with ammonium hydroxide.

Alternatively, the AgCNPS are produced via a method comprising (i) dissolving cerium and silver precursor salts such as cerium and silver nitrates; (ii) oxidizing and precipitating the dissolved cerium and silver precursor salts via admixture with ammonium hydroxide; (iii) washing and resuspending precipitated nanoparticles in water; (iv) subjecting the resuspended nanoparticles with hydrogen peroxide; and (v) washing the nanoparticles from step (iv) to remove ionized silver.

### Applications

The AgCNP2 can be combined into a composite with many forms of dental resins or sealants that may be applied to the teeth or the back teeth such as, without limitation, premolars and molars. Premolars may include maxillary first premolar, maxillary second premolar, mandibular first premolar and mandibular second premolar. Molars may include first molars, second molars and third molars. Third molars are known as wisdom teeth which appear generally between 17 and 21 years of age, but may be removed earlier by surgery.

Therefore, in some embodiments, multiple applications of a dental resin composite may be needed. The resin or sealant may be applied to a non-permanent teeth and then subsequently, applied to permanent teeth.

There are many dental resins and sealants readily available on the market. In some embodiments, the dental appropriate resin composite may be a light-curable resin composite. The dental resin composite is applied in a therapeutically effective amount to coat the teeth. The amount of AgCNP2 is mixed or dissolved in the dental resin composite in an amount that is a therapeutically effective amount.

Example resin composites are described in U.S. Pat. No. 4,826,893, entitled “Dental Resin Composition,” to Yamazaki et al., incorporated herein by reference in its entirety.

In some embodiments, acrylic-based polymer resins such as methyl methacrylate-based resin systems that include other monomer polymers and fillers are appropriate types of resin composites.

A methyl methacrylate-based dental resin may include a methyl methacrylate polymer resin (heat cured or self-cured or light cured) such as polymethyl methacrylate, a polymethyl methacrylate curing process that is initiated by tertiary and amine compounds.

The methyl methacrylate-based dental resin may include monomer polymers and fillers where a poly methyl methacrylate (PMMA) powder incorporates a filler such as silica, titania, or zirconia, and includes an initiator such as benzoyl peroxide. The methyl methacrylate-based dental resin may include a liquid component containing methyl methacrylate (MMA) monomer, with a crosslinking agent and inhibitor, where a combination of liquid with powder components initiates polymerization.

The methyl methacrylate-based dental resin may include PMMA powder, methacrylate monomer polymers and fillers such as titania and silica.

Another example resin composite is described in Published Application WO/201701886, entitled “Photopolymerisable Resin Composite and Use Thereof,” to DeOliveira, et al., incorporated herein by reference in its entirety. For example, the dental resin comprises a photopolimerisable resin composite with a higher filler particle content and a resin matrix of dimethacrylate monomers which are polymerised by free radical reaction initiated by the synergy of photoinitiator systems based on camphorquinone and (2,4,6-trimethylbenzoyl)diphenylphosphine oxide with monomer systems such as Bisphenol A Glycyl Dimethacrylate (Bis-GMA), Urethane Dimethacrylate (UDMA) Hydroxyethyl Methacrylate Phosphate (HEMA-P), glycerol dimethacrylate dihydrogen phosphate and mixtures of similar monomers.

A UDMA-based dental resin may include a photopolimerisable resin composite such as UDMA with a higher filler particle content and a resin matrix of dimethacrylate monomers, which are polymerised by free radical reaction initiated by the synergy of photoinitiator systems based on camphorquinone and (2,4,6-trimethylbenzoyl)diphenylphosphine oxide.

In some embodiments, Urethane Dimethacrylate-based dental resins (that can include other monomers and fillers) may be used, as described in A. Szczesio-Wlodarczyk et al., “An Evaluation of the Properties of Urethane Dimethacrylate-Based Dental Resins,” www.mdpi.com/1996-1944/4/1/2727/htm, incorporated herein by reference in its entirety.

A urethane dimethacrylate-based dental resin is light curable and may include UMDA combined with ethoxylated bisphenol-A dimethacrylate, for example.

Nanoparticles have been known to improve impact strength of dental acrylic resins. [Shcherbakov et al., entitled “Ce02 Nanoparticles-Containing Polymers for Biomedical Applications: A Review, 17 Mar. 2021, Polymers 2021, 3, 924, www.doi.org/10.3390/polym3060924.]

### Treatment/Prevention

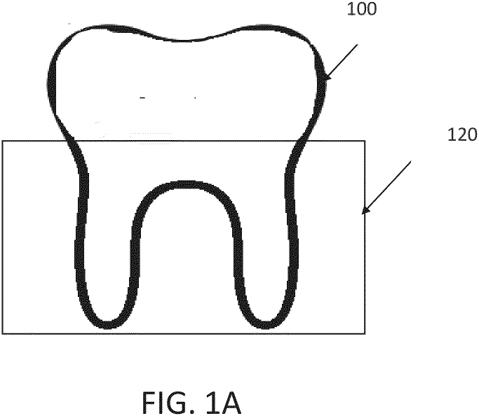

The method of preventing dental caries will be described in relation to FIGS. 1A, 1B and 2-4. The method of preventing dental caries may include forming a dental resin composite with AgCNP2 in the amount of about 0.01-0.1 wt. %. The method may include cleaning a subject’s teeth to remove surface contaminates. In some embodiments, surface contaminants may include plaque. Surface contaminants may include food particles, which may be embedded in the depressions or grooves naturally occurring or worn in the surface of a tooth or between surfaces of adjacent teeth.

FIG. 1A illustrates a subject tooth 100 in a clean state. The tooth 100 is represented in the gums 120 of a subject. FIG. 1B illustrates a subject tooth 100 of FIG. 1A with a coating 115 of dental resin composite including AgCNP2 (e.g., mCNPs 117), coated on the tooth 100. Specifically, the dental resin composite including AgCNP2 may be coated on the enamel of the tooth which is the outer surface of the tooth above the gums.

After the tooth surface (i.e., enamel) has been cleaned, the dental resin composite including AgCNP2 may be coated on the enamel of the tooth 100. In some embodiments, the coating 115 of the composite is cured. For example, the dental resin composite may be light cured. For example, an ultraviolet light (UV) source may be used. The curing process hardens the dental resin composite including AgCNP2 on the tooth’s surface.

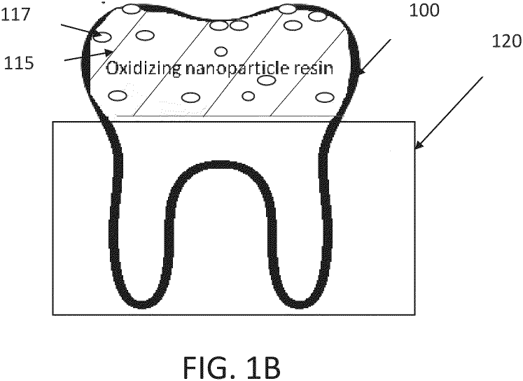

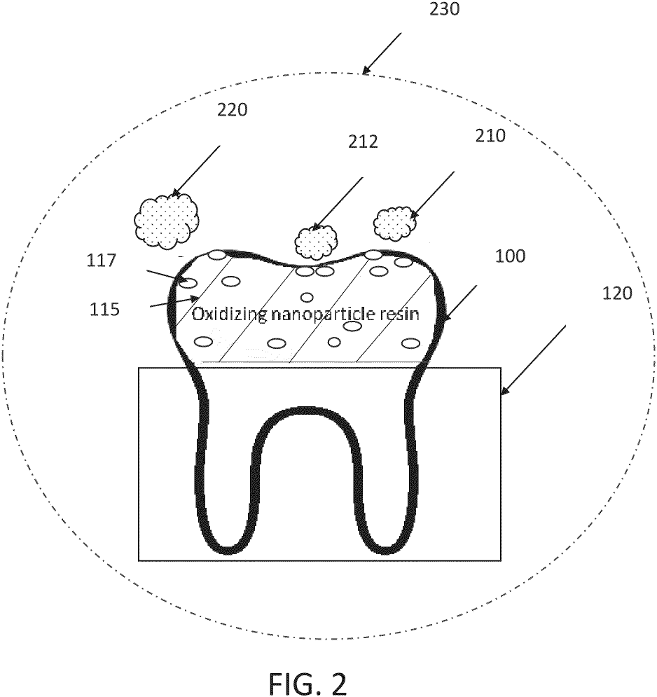

FIG. 2 illustrates a coated subject tooth 100 of FIG. 1B in the mouth 230 with bacteria 210, 212 and 220. FIG. 3 illustrates a coated subject tooth 100 of FIG. 2 releasing directed hydrogen peroxide (H₂O₂) to degrade or destroy caries-causing bacteria, denoted by 310 and 312. Bacteria that cause dental caries may include bacteria that feed on sugar, including carbohydrates that are left in the mouth. This bacteria may include, without limitation, streptococcus mutans, such as Streptococcus sobrinus, and lactobacilli acidophilus. The inventor has determined covering the tooth with cured dental resin composite with the metal-modified cerium oxide nanoparticles (mCNPs) 117, described herein, causes the autonomous release, from the mCNPs, hydrogen peroxide directed against bacteria in an oral cavity to prevent local acidification of the tooth and prevents further decay. The release of hydrogen peroxide is a non-indiscriminate release in the body and generally limited to release for destroying bacteria having a propensity to colonize on teeth.

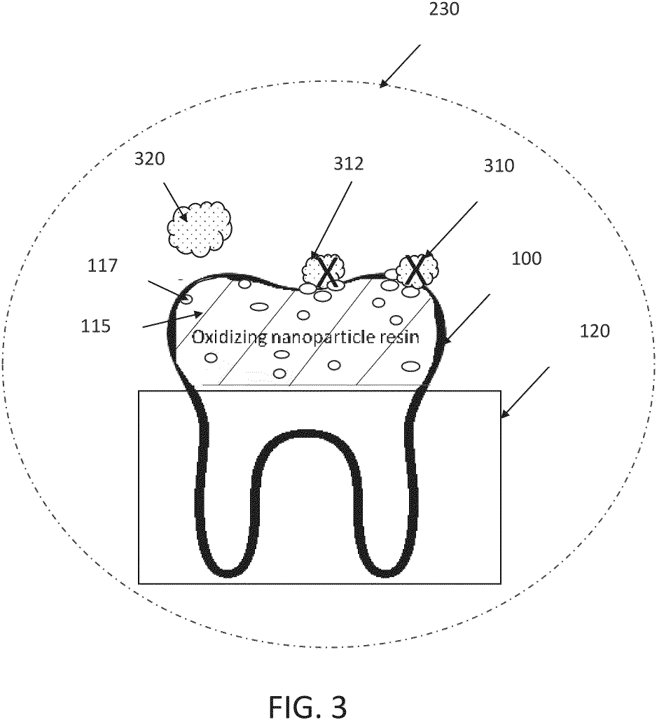

FIG. 4 illustrates the coated subject tooth 100 of FIG. 3 with the bacteria 310 and 312 degraded or destroyed. The bacteria 420 may be a different type of bacteria that does not have a propensity to colonize on teeth. Therefore, the mCNPs 117, as described herein, may not release hydrogen peroxide to destroy bacteria that do not make sustained contact with a treated tooth 420.

The method of prevention may have a synergic effect to treat the bacteria that may cause dental caries in an oral cavity that also causes other disorders, diseases and conditions in the human body.

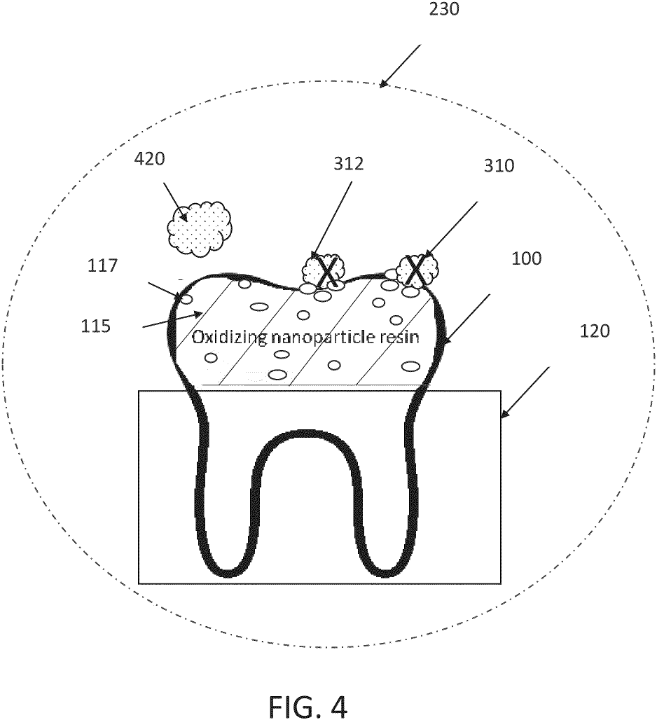

FIG. 5 illustrates a graphical representation 500 an efficacy test of AgCNP2 against Streptococcus mutans. The test included dried film including AgCNP2 (50 µg/ml) efficacy against Streptococcus mutans, which is commonly found in the human oral cavity and is a significant contributor to tooth decay. Bacteria was incubated with AgCNP2 for 1 hour at 37° C. (mimicking body temperature) showing eradication of the bacteria. The working medium was Tris/NaCl + 0.2% glycerol to ensure bacteria survival for the control.

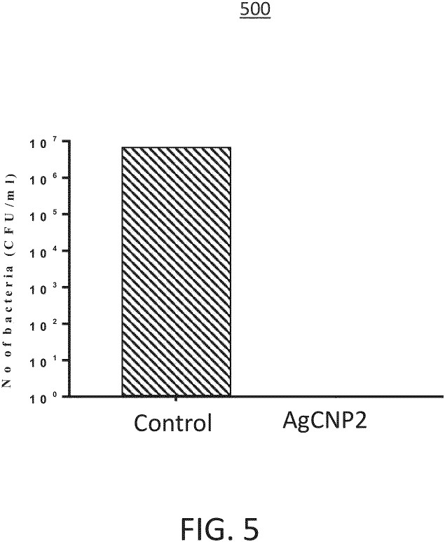

FIG. 6A illustrates a photograph 600A of a control surface in a pediatric facility that was not cleaned, and after 28 days P. aeruginosa, K. pneumoniae and S. aureus are present on the surface.

FIG. 6B illustrates a photograph 600B of an AgCNP2 with binder treated surface (<0.1 weight %) (“NanoRAD”) and the untreated surface, where neither were cleaned for 28 days, where the NanoRAD treated surface prevented bacteria survival for 28 days, and the untreated surface matched the control surface.

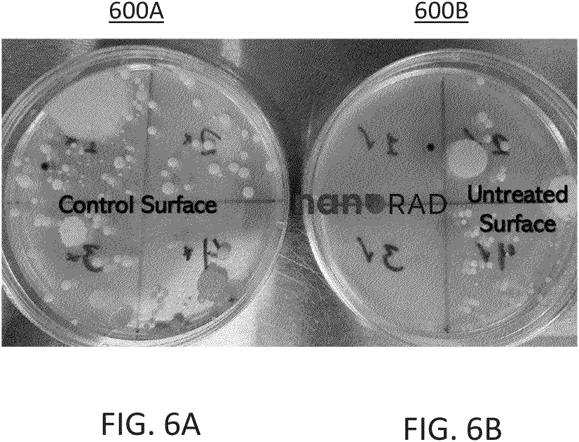

FIG. 7 illustrates a graphical representation 700 of efficacy tests of AgCNP2 (NanoRAD) at different incubation times with Escherichia coli (E. coli). The efficacy tests were evaluated at times 10, 30, 60 and 120 minutes to measure the colony-forming unit (CFU). No bacterial survival at any time point was noted between 10 minutes to 120 minutes for the NanoRAD of AgCNP2 with an AgCNP2 of 0.1 mg/ml or <0.01 weight% for the challenge.

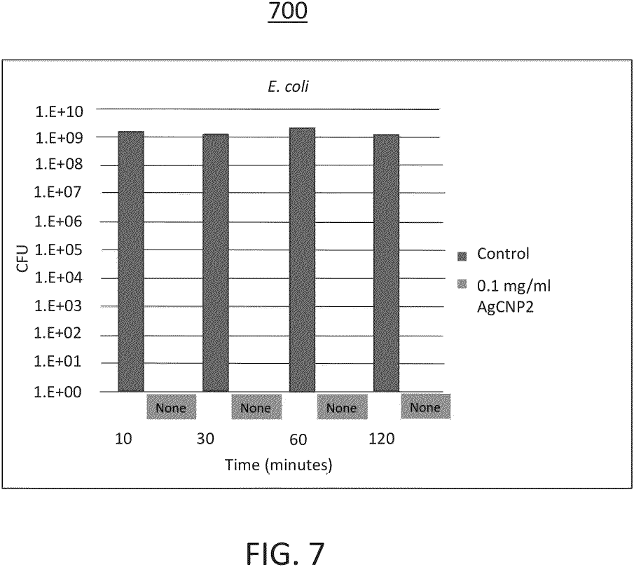

FIG. 8 illustrates a graphical representation 800 of efficacy tests of AgCNP2 (NanoRAD) at different incubation times with Staphylococcus epidermidis (S. epidermis). The efficacy tests were conducted at 10, 30, 60 and 120 minutes. The efficacy tests of a control substance are represented in a blue bar at 10, 30, 60 and 120 minutes. The efficacy test of the AgCNP2 evaluated at time = 10 minutes is shown in an orange bar. The efficacy tests of the AgCNP2 at times 30, 60 and 120 minutes was essentially 0 with an AgCNP2 of .1 mg/ml or <0.01 weight% for the challenge.

In FIGS. 7 and 8, the AgCNP2 (at 0.1 wt%) was mixed in a binder with silica and alumina and dispersant to form a coating composition with the AgCNP2 and then dried on glass.

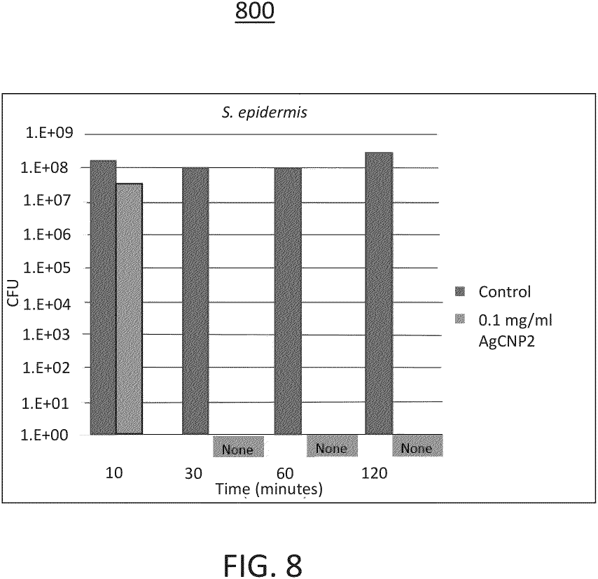

FIG. 9A illustrates a photograph 900 of S. epidermidis dried on glass and treated with a positive control medium in three separate areas/sections 1, 2 and 3. FIG. 9B illustrates a table 950 of test results over two hours of the S. epidermidis dried on the glass in areas/sections 1, 2 and 3. The CFUs/mL in plate sections 1 and 2 were found to be 7.0 × 10⁶ after two hours. The CFUs/mL in plate section 3 was found to be 9.0 × 10⁶ after two hours.

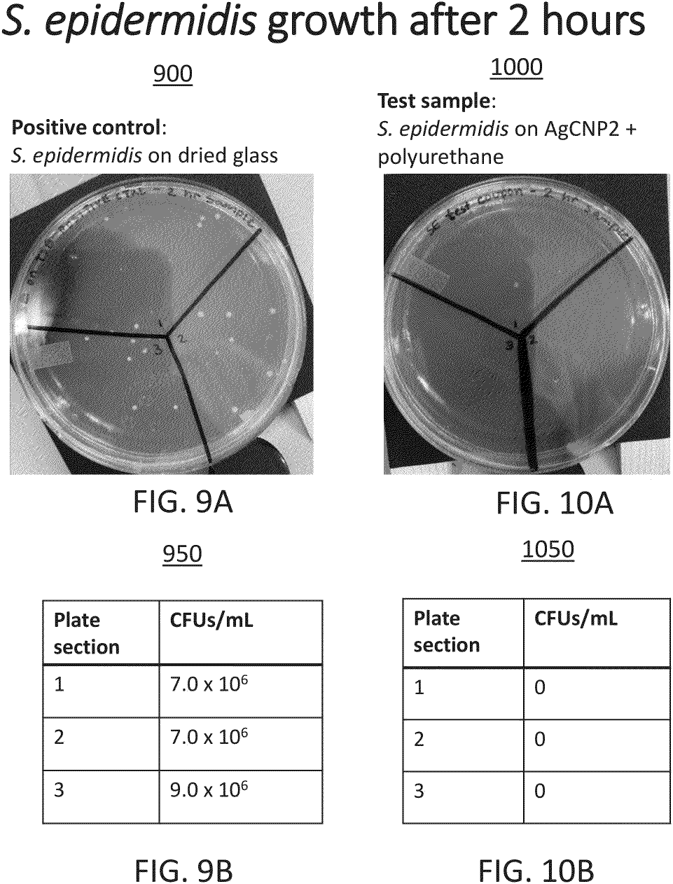

FIG. 10A illustrates a photograph 1000 of S. epidermidis dried on glass coated with AgCNP2 and polyurethane in three separate areas/sections 1, 2 and 3. FIG. 10B illustrates a table 1050 of test results over two hours of the S. epidermidis dried on the glass coated with AgCNP2 and polyurethane in three separate areas/sections 1, 2 and 3. The CFUs/mL in plate sections 1, 2 and 3 were found to be essentially 0 after two hours.

In the test of FIG. 10A, the AgCNPs were in a polyurethane matrix with nano-alumina (opaque filler for dental) and is fairly close polymer wise to an actual dental resin composite as a predictor of efficacy.

Two tests were also performed by MB Research Laboratories, in Spinnerstown, PA, using the AgCNP2 described herein. The tests include a skin irritation test (SIT) and an eye irritation test (EIT). The skin irritation test (SIT) and an eye irritation test (EIT) are used to predict the skin irritation of AgCNP2 to the skin in the oral cavity

### Skin Irritation Test (SIT) and Results

MatTek EpiDerm™ tissue samples were treated with the test articles, negative control, and positive control in triplicate for 60 minutes. Following treatment and subsequent incubation time, the viability of the tissues was determined using thiazolyl blue tetrazolium bromide (MTT) uptake and reduction. The absorbance of each sample was measured at 570 nm. The viability was then expressed as a percent of control values. If the mean tissue viability was 50% or less, the test material was classified as an irritant; if the mean tissue viability was more than 50%, the test material was classified as a non-irritant. The test used MB Protocol Number: 713-03.

Table 2 below identifies the test and control articles, the mean tissue viability percentage (%), the irritancy classification and the GHS classification. The purpose of this study was to provide classification of the dermal irritation potential of chemicals by using a three-dimensional human epidermis model, according to the OECD Guideline for the Testing of Chemicals No. 439, “In Vitro Skin Irritation: Reconstructed Human Epidermis Test Method”. [OECD: Organisation for Economic Co-Operation and Development.] The EpiDerm™ SIT allows discrimination between irritants (Category 2) and non-irritants, in accordance with U.N. GHS classification. [U.N. GHS: United Nations Globally Harmonized System of Classification and Labelling of Chemicals.]

TABLE 2

| Test and Control Article Identity | Mean Tissue Viability (%) | Irritancy Classification | GHS Classification |
|---|---|---|---|
| 0.073 wt% AgCNP2 in water | 112.4 | Non-irritant | No Category |
| 0.01 wt% AgCNP2 in water | 105.8 | Non-irritant | No Category |
| Dulbecco’s Phosphate-buffered saline (dPBS) (Negative Control) | 100.0 | Non-irritant | No Category |
| 5% Sodium dodecyl sulfate (Positive Control) | 3.1 | Irritant | Category 2 |

Table 3 shows a table of positive controls.

TABLE 3

Positive Control

| Identity | 5% Sodium dodecyl sulfate (SDS), Lot No. 111120BBB |
|---|---|
| Test Article Characterization | |
| Description | Clear colorless liquid |
| Sample Preparation | Used as received |

Table 4 shows a table of negative controls.

TABLE 4

Negative Control

| Identity | Dulbecco’s Phosphate-buffered saline (dPBS) Lot No. 2306404 |
|---|---|
| Test Article Characterization | |
| Description | Clear colorless liquid |
| Sample Preparation | Used as received |

### Plate Reader Linearity Check

The linearity of the plate reader used for optical density (OD) determination was verified prior to its use the same week the SIT assay was performed. A dilution series of trypan blue was prepared and two 200-µl aliquots per concentration were pipetted into a 96-well plate. The optical density of the plate wells was measured at a wavelength of 570 nm (OD570), with no reference wavelength. A regression line and an R-squared value were generated using Microsoft Excel® 2007. Verification was considered acceptable if the R-squared value was greater than or equal to 0.999.

### Test Article Reduction of MTT

For each test article, a total of 50 µl of the test article were mixed with 1 ml of MTT solution (1 mg/ml methyl thiazole tetrazolium diluted in Dulbecco’s Modified Eagle’s Medium [DMEM]). A negative control (50 µl of tissue culture water, TCH2O) was tested concurrently. The solutions were incubated in the dark at 37 ± 1° C., 5 ± 1% CO2 for approximately 3 hours in a six-well plate. After incubation, the solutions were visually inspected for purple coloration, which indicates that the test article reduced MTT. Since tissue viability is based on MTT reduction, direct reduction by a test article can exaggerate viability, making a test article seem less irritating than its actual irritation potential. None of the test articles were found to have reduced MTT and the assay continued as per the protocol.

### Mesh Compatibility

For each test article, pre-cut nylon mesh supplied with the tissues was placed on a slide and 30 µl of the undiluted test article or tissue culture water (negative control) were applied. After 60 minutes of exposure, the mesh was checked microscopically. No interaction between any test articles or tissue culture water and the mesh was observed so the test articles were dosed using the mesh as a spreading aid.

### Assessment of Coloring or Staining Materials

The test articles were non-colored; therefore, it was assessed to determine if the extractant would become colored when mixed with the test article. For each test article, a total of 50 µl of the test article were incubated in a six-well plate with 1 ml of TCH2O for at least one hour in a humidified 37 ± 1° C., 5 ± 1% CO2 incubator. An additional 50 µl of the test article were added to 2 ml of extractant (isopropanol) and incubated for 2 to 3 hours in a six-well plate, at room temperature with shaking. Two 200-µl aliquots of the test article plus TCH2O or test article plus extractant from each well were transferred to a 96-well plate and measured at 570 nm using a plate reader (µQuant Plate Reader, Bio-Tek Instruments, Winooski, VT). No color developed in the water or the extractant, resulting in an OD570 no more than 0.08 after subtraction of blank (TCH2O or isopropanol, respectively), so no colorant controls were added to the assay.

### EpiDerm™ Tissue Samples

EpiDerm™ tissues, Lot No. 35611, Kit F, were received from MatTek Corporation (Ashland, MA) on 07 Jul. 2021 and refrigerated at 2 to 8° C. Before use, the tissues were incubated (37 ± 1° C., 5 ± 1% CO2) with assay medium (MatTek) for a one- hour equilibration. The tissues were then moved to new wells with fresh medium for an additional overnight equilibrium, for 18 ± 3 hours. Equilibration medium was replaced with fresh medium before dosing.

### Dosing

Each treatment with the test articles or controls was conducted in triplicate. For each test article, 30 µl of the test article were applied to each EpiDerm™ tissue. A nylon mesh was then placed on top to facilitate even distribution of the test article.

A negative control (30 µl of Dulbecco’s Phosphate-buffered saline) and a positive control (30 µl of 5% SDS solution) were each tested concurrently, with a nylon mesh placed on top to facilitate even distribution of the material. The exposure period for the test articles and controls was 60 minutes. The dosed tissues were placed in an incubator at 37 ± 1° C., 5 ± 1% CO2 for 35 ± 1 minute, and then returned to the sterile hood for the remainder of the 60-minute exposure period.

After dosing and incubation, the tissues were rinsed with DPBS, blotted to remove the test substance, and dry the tissue, and transferred to fresh medium. The rinsed EpiDerm™ tissues were returned to the incubator for 24 ± 2 hours. Medium was changed at 24 ± 2 hours. Tissues were returned to the incubator for an additional 18 ± 2 hours.

### Tissue Viability (MTT Reduction)

Each EpiDerm™ tissue was transferred to a 24-well plate containing 300 µl of MTT solution (1 mg/ml MTT in DMEM). The tissues were then returned to the incubator for an MTT incubation period of 3 hours ± 10 minutes. Following the MTT incubation period, each EpiDerm™ tissue was rinsed with DPBS and then treated with 2.0 ml of extractant solution (isopropanol) per well for at least two hours, with shaking, at room temperature. Two 200-µl aliquots of the extracted MTT formazan were transferred to a 96-well plate and measured at 570 nm using a plate reader (µQuant Plate Reader, Bio-Tek Instruments, Winooski, VT).

### Quality Controls

The assay met the acceptance criteria if the mean OD570 of the negative control tissues was between and 2.8, inclusive, and the mean viability of positive control tissues, expressed as percentage of the negative control tissues, was less than or equal to 20%. In addition, the difference calculated from individual percent tissue viabilities of the three identically-treated replicates was acceptable if it was less than 18%.

### Skin Irritation Prediction

According to the EU and GHS classification, an irritant is predicted if the mean relative tissue viability of three individual tissues exposed to the test substance is 50% or less of the mean viability of the negative controls. [EU: OECD Guideline for the Testing of Chemicals No. 439: In Vitro Skin Irritation: Reconstructed Human Epidermis Test Method; and European Centre for the Validation of Alternative Methods (ECVAM) Joint Research Centre (ESAC), Statement on the Scientific Validity of In-Vitro Tests for Skin Irritation Testing.] [GHS: Globally Harmonized System of Classification and Labeling of Chemicals (GHS), 8th revised edition, 2019. United Nations – New York and Geneva.]

Table 5 identifies the irritancy classification and GHS category.

TABLE 5

| In Vitro Result | In Vivo Prediction | |
|---|---|---|
| | Irritancy Classification | GHS |
| Mean tissue viability ≤ 50% | Irritant | Category 2 |
| Mean tissue viability > 50% | Non-Irritant | No Category |

Each tissue in this group had individual relative viabilities greater than 50%, making each tissue non- irritating.

Table 6A is part 1 of a live tissue data table. The table identifies the tissue number to raw data test sample Aliq. 1, Aliq. 2, Aliquots, and mean blank. Table 6B is part 2 of the live tissue data table of Table 5 and identifies the corrected data, mean of Aliquots, OD means and differential, and viabilities both mean and standard deviation.

TABLE 6A

| Test and Control Article Identity | Tissue No. | Raw data | | Mean Blank |
|---|---|---|---|---|
| | | Aliq. 1 | Aliq. 2 | |
| 0.073 wt% AgCNP2 in water | 1 | 1.870 | 2.307 | 0.047 |
| | 2 | 2.189 | 2.154 | |
| | 3 | 1.722 | 1.698 | |
| 0.01 wt% AgCNP2 in water | 1 | 1.857 | 2.108 | 0.047 |
| | 2 | 1.867 | 1.826 | |
| | 3 | 1.789 | 1.806 | |
| Dulbecco’s Phosphate-buffered saline (dPBS) (Negative Control) | 1 | 1.861 | 1.752 | 0.047 |
| | 2 | 1.600 | 1.652 | |
| | 3 | 1.939 | 1.850 | |
| 5% Sodium dodecyl sulfate (SDS) (Positive Control) | 1 | 0.091 | 0.112 | 0.047 |

TABLE 6B

| Test and Control Article Identity | Tissue No. | Corrected Data | | Mean of Aliquo ts | % Viabili ty | OD | | Viabilities(%) | |
|---|---|---|---|---|---|---|---|---|---|
| | | Aliq. 1 | Aliq. 2 | | | Mean | Diff. | Mean | SD |
| 0.073 wt% AgCNP2 in water | 1 | 1.823 | 2.260 | 2.042 | 118.1 | 1.943 | 0.246 | 112.4 | 14.2 |
| | 2 | 2.142 | 2.107 | 2.125 | 122.9 | | | | |
| | 3 | 1.675 | 1.651 | 1.663 | 96.2 | | | | |
| 0.01 wt% AgCNP2 in water | 1 | 1.810 | 2.061 | 1.936 | 112.0 | 1.829 | 0.096 | 105.8 | 5.5 |
| | 2 | 1.820 | 1.779 | 1.800 | 104.1 | | | | |
| | 3 | 1.742 | 1.759 | 1.751 | 101.3 | | | | |
| Dulbecco’s Phosphate-buffered saline (dPBS) (Negative Control) | 1 | 1.814 | 1.705 | 1.760 | 101.8 | 1.729 | 0.137 | 100.0 | 7.9 |
| | 2 | 1.553 | 1.605 | 1.579 | 91.3 | | | | |
| | 3 | 1.892 | 1.803 | 1.848 | 106.9 | | | | |
| 5% Sodium dodecyl sulfate (SDS) (Positive Control) | 1 | 0.044 | 0.065 | 0.055 | 3.2 | 0.054 | 0.003 | 3.1 | 0.1 |
| | 2 | 0.052 | 0.050 | 0.051 | 3.0 | | | | |
| | 3 | 0.059 | 0.053 | 0.056 | 3.2 | | | | |

Table 7 identifies the OD blank values.

TABLE 7

| | | | | | | | |
|---|---|---|---|---|---|---|---|
| Tissue: | 1 | 2 | 3 | 4 | 5 | 6 | Mean Blank OD |
| OD: | 0.048 | 0.046 | 0.046 | 0.048 | 0.047 | 0.046 | 0.047 |

### Quality Controls

The mean OD570 of the negative control tissues was 1.729, which met the acceptance criteria of greater than or equal to 0.8 and less than or equal to 2.8. The mean relative viability of the positive control tissues was 3.1%, which met the acceptance criterion of less than or equal to 20%. The standard deviation in viability between identically treated tissues were 0.1% to 19.6%, which did not meet the acceptance criterion of less than 18%. The R-squared value calculated for the plate reader linearity check was 0.9997, which met the acceptance criterion of greater than or equal to 0.999.

Table 8 identifies the PBS. Dulbeccos W/O CA. MG (1X) with an origin of the UK.

TABLE 8

| | | | |
|---|---|---|---|
| Test | Specification | Result | Units |
| a01 Bacterial | Negative | Negative | |
| a02 Fungal | Negative | Negative | |
| a96 Endotoxin | 0.000 to 0.500 | 0.040 | EU/mL |
| b01 Ph | 7.00 to 7.30 | 7.17 | pH Units |
| b02 Osmolality | 270 to 300 | 283 | mOs/kg H2O |
| b33 Volume | Satisfactory | Satisfactory | |
| q01 Documentation | Satisfactory | Satisfactory | |
| q02 Appearance (Liquid) | Satisfactory | Satisfactory | |
| Sterile filtered (0.1 µm); Sterility testing (Test Numbers a01, a02) is carried out in accordance with Ph.Eur. 2.6.1 and USP 7.1 | | | |

### Eye Irritation Test (EIT) and Results

MatTek EpiOcular™ tissues were treated with the test articles, negative control, and positive control in duplicate for 30 minutes. Following treatment and subsequent incubation time, the viability of the tissues was determined using thiazolyl blue tetrazolium bromide (MTT) uptake and reduction. The absorbance of each sample was measured at 570 nm. The viability was then expressed as a percent of negative control values. If the mean tissue viability were less than or equal to 60%, the test material was classified as an Irritant (UN GHS Category 1 or 2); if the mean tissue viability were greater than 60%, the test material was classified as UN GHS No Category and was, therefore, interpreted to be Non-irritant. The MB protocol: 772.

Table 9 shows the irritancy classification and GHS classification, as well as the tissue viability for mean and difference for each test article.

The summarized results and irritation classifications are as follows, as shown in Table 9:

TABLE 9

| Test and Control Article Identity | Tissue Viability (%) | | Irritancy Classification | GHS Classification |
|---|---|---|---|---|
| | Mean | Diff. | | |
| Test Article 1: 0.073 wt% AgCNP2 in water | 96.8 | 13.47 | Non-Irritant | No Category |
| Test Article 2: 0.01 wt% AgCNP2 in water | 105.2 | 4.61 | Non-Irritant | No Category |
| Tissue Culture Water (Run 1) (Negative Control) | 100.0 | 2.51 | Non-Irritant | No Category |
| Methyl acetate (Run 1) (Positive Control) | 30.1 | 4.28 | Irritant | Category 1 or 2 |
| Tissue Culture Water (Run 2) (Negative Control) | 100.0 | 5.63 | Non-Irritant | No Category |
| Methyl acetate (Run 2) (Positive Control) | 40.9 | 1.28 | Irritant | Category 1 or 2 |
| Diff. = difference between tissues | | | | |

The purpose of this study was to provide classification of chemicals concerning their eye irritation potential using an alternative to the Draize Rabbit Eye Test, according to the OECD Test Guideline No. 492, “Reconstructed Human Cornea-like Epithelium (RhCE) Test Method for Identifying Chemicals Not Requiring Classification and Labelling for Eye Irritation or Serious Eye Damage.” The EpiOcular™ EIT was intended to differentiate those materials that are UN GHS No Category (i.e., do not meet the requirements for UN GHS classification) from those that would require labeling as either UN GHS Category 1 or 2.

### Limitation

This assay was not intended to differentiate between UN GHS Category 1 and UN GHS Category 2 (nor between EU R36 and R41).

Table 10 lists a description of the test articles.

TABLE 10

| | | |
|---|---|---|
| Identity | Description | Sample Preparation |
| Test Article 1: 0.073 wt% AgCNP2 in water | Clear Colorless Liquid | Used as received |
| Test Article 2: 0.01 wt% AgCNP2 in water | Clear Colorless Liquid | Used as received |

Table 11 lists a description of the Positive control and Table 12 lists a description of the Negative Control.

TABLE 11

Positive Control

| | |
|---|---|
| Identity | Methyl acetate, Lot No. 060321JKA (Characterization not provided by supplier) |
| Storage | Room temperature and humidity |
| Description | Clear colorless |
| Provided by | MatTek Corporation |

TABLE 12

Negative Control

| | |
|---|---|
| Identity | Tissue Culture Water, (TCH2O) Lot No. RNBJ0203 |
| Storage | Room temperature and humidity |
| Description | Clear colorless |
| Provided by | Sigma-Aldrich® |

### Plate Reader Linearity Check

The linearity of the plate reader used for optical density (OD) determination was verified prior to its use the same week the EIT assay was performed. A dilution series of trypan blue was prepared and two 200- µl aliquots per concentration were pipetted into a 96-well plate. The optical density of the plate wells was measured at a wavelength of 570 nm (OD570), with no reference wavelength. A regression line and an R-squared value were generated using Microsoft Excel® 2007. Verification was considered acceptable if the R-squared value was greater than 0.999.

### Test Article Reduction of MTT

For each test article, A total of 50 µl of the test article were mixed with 1 ml of MTT solution (1 mg/ml methyl thiazole tetrazolium diluted in Dulbecco’s Modified Eagle’s Medium [DMEM]). A negative control (50 µl of tissue culture water, TCH2O) was tested concurrently. The solutions were incubated in the dark at 37 ± 1° C., 5 ± 1% CO2 for approximately 3 hours in a six-well plate. After incubation, the solutions were visually inspected for purple coloration, which indicates that the test article reduced MTT. Since tissue viability is based on MTT reduction, direct reduction by a test article can exaggerate viability, making a test article seem less irritating than its actual irritation potential. None of the test articles were found to have reduced MTT and the assay continued as per the protocol.

### Assessment of Coloring or Staining Materials

The test articles 0.073 wt% AgCNP2 in water and 0.01 wt% AgCNP2 in water were non-colored; therefore, they were assessed to determine if the extractant would become colored when mixed with the test article.

For each test article, a total of 50 µl of the test article were incubated in a six-well plate with 1 ml of TCH2O for at least one hour in a humidified 37 ± 1° C., 5 ± 1% CO2 incubator. An additional 50 µl of the test article were added to 2 ml of extractant (isopropanol) and incubated for 2 to 3 hours in a six-well plate, at room temperature with shaking. Two 200-µl aliquots of the test article plus TCH2O or test article plus extractant from each well were transferred to a 96-well plate and measured at 570 nm using a plate reader (µQuant Plate Reader, Bio-Tek Instruments, Winooski, VT). No color developed in the water or the extractant, resulting in an OD570 no more than 0.08 after subtraction of blank (TCH2O or isopropanol, respectively), so no colorant controls were added to the assay.

### EpiOcular™ Tissue Samples

EpiOcular™ tissues, Lot No. 31794, Kit C and A, were received from MatTek Corporation (Ashland, MA) on Jun. 29, 2021, and refrigerated at 2 to 8° C. Before use, the tissues were incubated (37 ± 1° C., 5 ± 1% CO2) with assay medium (MatTek) for a one-hour equilibration. Equilibration medium was replaced with fresh medium for an additional overnight equilibration of 16 to 24 hours. After the overnight incubation, the tissues were moistened with 20 µl of Dulbecco’s phosphate-buffered saline (DPBS) and incubated at 37 ± 1° C., 5 ± 1% CO2 for 30 ± 2 minutes.

### Dosing

For each test article, a total of 50 µl of the test article were applied to EpiOcular™ tissues. A negative control (50 µl of TCH2O) and a positive control (50 µl of methyl acetate) were each tested concurrently. Each treatment with test article or control was conducted in duplicate. The tissues were incubated at 37 ± 1° C., 5 ± 1% CO2 for 30 ± 2 minutes. After dosing and incubation, the tissues were rinsed with PBS and soaked in 5 ml of room-temperature assay medium in a 12-well plate for 12 ± 2 minutes. The soaked tissues were then incubated in fresh assay medium at 37 ± 1° C., 5 ± 1% CO2 for 120 ± 15 minutes.

### Tissue Viability (MTT Reduction)

At the end of the incubation period, each EpiOcular™ tissue was transferred to a 24-well plate containing 300 µl of MTT solution (1 mg/ml MTT in DMEM). The tissues were then returned to the incubator for an MTT incubation period of 3 hours ± 10 minutes. Following the MTT incubation period, each EpiOcular™ tissue was rinsed with DPBS and then treated with 2.0 ml of extractant (isopropanol) in a 24-well plate overnight at room temperature in the dark allowing extraction to occur through both the top and bottom of the insert. Two 200-µl aliquots of the extracted MTT formazan from each well were transferred to a 96-well plate and measured at 570 nm using a plate reader (µQuant Plate Reader, Bio- Tek Instruments, Winooski, VT).

### Analysis of Data

### Viability

See Tables 14A and 14B for Experimental Data. The mean absorbance value for each time point was calculated from the optical density (OD) of the duplicate samples and expressed as percent viability for each sample using the following formula Eq(1):

$$\% \text{viability = 100 X} \left(\text{ODsample / ODnegative control}\right)$$

### Quality Controls

The assay meets the acceptance criteria if the mean OD570 of the negative control tissues is greater than 0.8 and less than 2.5, and the mean relative viability of positive control tissues, expressed as percentage of the negative control tissues, is less than 50%. In addition, the difference in viability between identically treated tissues must be less than 20%.

### Ocular Irritation Prediction

According to the OECD Guideline, and GHS classification, an irritant is predicted if the mean relative tissue viability of two individual tissues exposed to the test substance is ≤ 60% of the mean viability of the negative controls. Table 13 shows the mean tissue viability for the GHS Classification.

TABLE 13

| | |
|---|---|
| In Vitro Result | GHS Classification |
| Mean tissue viability less than or equal to 60% | Category 1 or 2 |
| Mean tissue viability greater than 60% | No Category |

If the test article-treated tissue viability is 60 ± 5%, a second EIT should be performed. If the results of the second test disagree with the first, then a third test should be performed. The conclusion will be based on the agreement of two of the three tests.

Tables 14A and 14B show the experimental data, per test article. Table 14A shows the raw OD, the blank corrected OD data and mean of aliquots. Table 14B shows the percent viability per test article, the OD mean, OD difference, the viability % means and the viability % difference. Table 15A shows the OD blank data for run 1. Table 15B shows the OD blank data for run 2.

TABLE 14A

| Test and Control Article Identity | Tissue No. | Raw OD data | | Blank corrected OD data | | Mean of Aliquots |
|---|---|---|---|---|---|---|
| | | Aliq. 1 | Aliq. 2 | Aliq. 1 | Aliq. 2 | |
| Test Article 1: 0.073 wt% AgCNP2 in water (Run 1) | 1 | 2.096 | 2.330 | 2.049 | 2.283 | 2.166 |
| | 2 | 1.822 | 2.040 | 1.775 | 1.993 | 1.884 |
| Test Article 2: 0.01 wt% AgCNP2 in water (Run 1) | 1 | 2.179 | 2.220 | 2.132 | 2.173 | 2.153 |
| | 2 | 2.324 | 2.268 | 2.277 | 2.221 | 2.249 |
| TCH2O (Run 1) (Negative Control) | 1 | 2.092 | 2.240 | 2.045 | 2.193 | 2.119 |
| | 2 | 2.097 | 2.130 | 2.050 | 2.083 | 2.067 |
| Methyl acetate (Run 1) (Positive Control) | 1 | 0.636 | 0.628 | 0.589 | 0.581 | 0.585 |
| | 2 | 0.710 | 0.733 | 0.663 | 0.686 | 0.675 |
| TCH2O (Run 2) (Negative Control) | 1 | 2.354 | 2.141 | 2.308 | 2.095 | 2.201 |
| | 2 | 2.166 | 2.088 | 2.120 | 2.042 | 2.081 |
| Methyl acetate (Run 2) (Positive Control) | 1 | 0.934 | 0.939 | 0.888 | 0.893 | 0.890 |
| | 2 | 0.911 | 0.907 | 0.865 | 0.861 | 0.863 |

TABLE 14B

| Test and Control Article Identity | Tissue No. | % Viability | OD | | % Viabilities | |
|---|---|---|---|---|---|---|
| | | | Mean | Diff. | Mean | Diff. |
| Test Article 1: 0.073 wt% AgCNP2 in water (Run 1) | 1 | 103.5 | 2.025 | 0.282 | 96.8 | 13.47 |
| | 2 | 90.0 | | | | |
| Test Article 2: 0.01 wt% AgCNP2 in water (Run 1) | 1 | 102.9 | 2.201 | 0.096 | 105.2 | 4.61 |
| | 2 | 107.5 | | | | |
| | 2 | 9.4 | | | | |
| TCH2O (Run 1) (Negative Control) | 1 | 101.3 | 2.093 | 0.053 | 100.0 | 2.51 |
| | 2 | 98.7 | | | | |
| Methyl acetate (Run 1) (Positive Control) | 1 | 28.0 | 0.630 | 0.089 | 30.1 | 4.28 |
| | 2 | 32.2 | | | | |
| TCH2O (Run 2) (Negative Control) | 1 | 102.8 | 2.141 | 0.121 | 100.0 | 5.63 |
| | 2 | 97.2 | | | | |
| Methyl acetate (Run 2) (Positive Control) | 1 | 41.6 | 0.877 | 0.028 | 40.9 | 1.28 |
| | 2 | 40.3 | | | | |
| Diff. = difference between tissues OD = optical density | | | | | | |

TABLE 15A

Blank Data
Run 1

| | | | | | | | | | |
|---|---|---|---|---|---|---|---|---|---|
| No. | 1 | 2 | 3 | 4 | 5 | 6 | 7 | 8 | Mean |
| OD | 0.47 | 0.47 | 0.46 | 0.46 | 0.46 | 0.47 | 0.46 | 0.48 | 0.47 |

TABLE 15B

Run 2

| | | | | | | | | | |
|---|---|---|---|---|---|---|---|---|---|
| No. | 1 | 2 | 3 | 4 | 5 | 6 | 7 | 8 | Mean |
| OD | 0.46 | 0.46 | 0.46 | 0.46 | 0.46 | 0.46 | 0.46 | 0.47 | 0.46 |

### Quality Controls

The mean OD570 of the negative control tissues were 2.093 and 2.141, which met the acceptance criteria of greater than 0.8 and less than 2.5. The mean relative viabilities of the positive control tissues were 30.1% and 40.9%, which met the acceptance criterion of less than 50%. The differences in viability between identically treated tissues were 1.03% to 13.47%, which met the acceptance criterion of less than 20%. The R-squared value calculated for the plate reader linearity check was 0.9999, which met the acceptance criterion of greater than 0.999. All controls passed the acceptance criteria for a valid study.

In view of the foregoing, the embodiments herein are directed to a dental resin composition comprising metal-modified cerium oxide nanoparticles (mCNPs), as described herein, and a dental resin, the metal-modified cerium oxide nanoparticles (mCNPs) is in an amount ranging from about 0.01 - 0.1% by weight.

In some embodiments, the dental resin composition comprising metal-modified cerium oxide nanoparticles (mCNPs), as described herein, and a dental resin, the mCNPs releases directed hydrogen peroxide that is then used against bacteria in an oral cavity. This prevents local acidification of the tooth and prevents further decay.

Moreover, the hydrogen peroxide is not indiscriminately released into the body.

The dental resin composition may include a metal is selected from the group consisting of silver, gold, ruthenium, vanadium, copper, titanium, nickel, platinum, titanium, tin, zinc and iron. The metal may be an antimicrobial promoting metal that is non-ionizing meaning the metal is a non-ionic metal.

In some embodiments, the metal (m) of the dental resin composition comprises AgCNP2 in an amount of less than about 0.01 to 0.1% by weight in the resin composition.

The mCNPs of the resin composition are in the range of about 3-25 nm in size.

The mCNPs of the resin composition are in the range of about 3-35 nm in size.

The mCNPs of the resin composition may comprise a predominantly 3+ cerium surface charge. The predominately 3+ charge allows for the hydrogen peroxide reaction to dominate.

Moreover, the method may include treating teeth with the mCNPs, described herein, and/or a resin composition including the mCNPs in an amount of less than about 0.01 to 0.1% by weight in the resin composition to assist with a body’s naturally occurring processes to remineralize tooth material to further strengthen teeth against decay and sensitivity where the mCNPs in the resin composition have a predominantly 3+ cerium surface charge and are in the range of about 3-25 nm or 3-35 nm in size.

In some embodiments, the AgCNP2 of the resin composition is produced via a method comprising dissolving cerium and silver precursor salts such as cerium and silver nitrates and oxidizing the dissolved cerium and silver precursor salts. The silver (Ag) is a stable metallic silver that is non-ionizing.

In some embodiments, the AgCNP2 of the resin composition is produced via a method comprising dissolving cerium and silver precursor salts such ascerium and silver nitrates; oxidizing the dissolved cerium and silver precursor salts via admixture with peroxide; and precipitating nanoparticles by subjecting the admixture with ammonium hydroxide.

In some embodiments, the AgCNP2 of the resin composition is produced via a method comprising (i) dissolving cerium and silver precursor salts suchas cerium and silver nitrates; (ii) oxidizing and precipitating the dissolved cerium and silver precursor salts via admixture with ammonium hydroxide; (iii) washing and resuspending precipitated nanoparticles in water; (iv) subjecting the resuspended nanoparticles with hydrogen peroxide; and (v) washing the nanoparticles from step (iv) to remove ionized silver where the mCNPs in the resin composition have a predominantly 3+ cerium surface charge and are in the range of about 3-25 nm in size. Accordingly, the resultant metal (m) in the mCNP ingredient is a non-ionic metal with antimicrobial promoting properties.

In some embodiments, a method of preventing dental caries includes coating at least one tooth with a dental resin composition comprising metal-modified cerium oxide nanoparticles (mCNPs), as described herein, and a dental resin; and releasing from the mCNPs directed hydrogen peroxide that is then used against bacteria in an oral cavity to prevent local acidification of the tooth and prevents further decay where the mCNPs in the resin composition are in the range of about 3-25 nm or 3-35 nm in size.

Moreover, the methods herein may include non-indiscriminately releasing the hydrogen peroxide into the body, which is generally limited to release for destroying bacteria having a propensity to colonize on teeth.

In an embodiment, the method herein may include wherein the forming of the resin composition includes forming a dental resin that includes hot or cold cured methyl methacrylate-based polymer resin.

In an embodiment, the method herein may include wherein the forming of the resin composition includes forming a dental resin that includes a methyl methacrylate-based resin having PMMA powder, methacrylate monomer polymers and fillers that include titania and silica.

In an embodiment, the method herein may include wherein the forming the resin composition includes forming a dental resin that includes a urethane dimethacrylate-based dental resin or co-monomers with urethane dimethacrylate-based resins.

In an embodiment, the method herein may include wherein the forming the resin composition includes forming a dental resin that includes a photopolimerisable resin composite having urethane dimethacrylate with a filler particle content and a resin matrix of dimethacrylate monomers which are polymerised by free radical reaction initiated by synergy of a photoinitiator system based on camphorquinone and (2,4,6-trimethylbenzoyl)diphenylphosphine oxide.

In an embodiment, the method herein may include wherein forming the resin composition includes forming a dental resin that includes a liquid component containing methyl methacrylate (MMA) monomer with a crosslinking agent and inhibitor.

Moreover, the methods herein may include applying mCNPs, as described herein, to many forms of dental appropriate resins, such as acryl-based polymers, to assist with a body’s naturally occurring processes to remineralize tooth material to further strengthen teeth against decay from bacteria in the oral cavity and sensitivity where the mCNPs in the resin composition have a predominantly 3+ cerium surface charge and are in the range of about 3-25 nm in size and m is an antimicrobial promoting metal.

The terminology used herein is for the purpose of describing particular embodiments only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order or importance, but rather the terms first, second, etc., are used to distinguish one element from another. As used herein the expression “at least one of A and B,” will be understood to mean only A, only B, or both A and B.

Embodiments are described herein with reference to the attached figures wherein like reference numerals are used throughout the figures to designate similar or equivalent elements. In some instances, figures are not drawn to scale and they are provided merely to illustrate aspects disclosed herein.

While various disclosed embodiments have been described above, it should be understood that they have been presented by way of example only, and not limitation. Numerous changes, omissions and/or additions to the subject matter disclosed herein can be made in accordance with the embodiments disclosed herein without departing from the spirit or scope of the embodiments. Also, equivalents may be substituted for elements thereof without departing from the spirit and scope of the embodiments. In addition, while a particular feature may have been disclosed with respect to only one of several implementations, such feature may be combined with one or more other features of the other implementations as may be desired and advantageous for any given or particular application. Furthermore, many modifications may be made to adapt a particular situation or material to the teachings of the embodiments without departing from the scope thereof.

Therefore, the breadth and scope of the subject matter provided herein should not be limited by any of the above explicitly described embodiments. Rather, the scope of the embodiments should be defined in accordance with the following claims and their equivalents.

What is claimed is:

1. A dental resin composition comprising:
a dental resin; and
metal-modified cerium oxide nanoparticles (mCNPs) having a predominantly 3+ cerium surface charge and in a range of about 3-25 nm in size and mixed in an amount that is in a range of about 0.01 to 0.1 weight percentage of a mixture having the dental resin and the mCNPs, where m is an antimicrobial promoting metal that is non-ionizing, and the mCNPs effectuate release directed hydrogen peroxide (H₂O₂) in an oral cavity that is then used against bacteria in the oral cavity to prevent local acidification of a tooth on which a therapeutically effect amount of the mixture is applied and cured.

2. The dental resin composition according to claim 1, wherein the metal of the mCNPs is selected from the group consisting of silver, gold, ruthenium, vanadium, copper, titanium, nickel, platinum, titanium, tin, zinc and iron.

3. The dental resin composition according to claim 1, wherein the metal of the mCNPs is a stable metallic silver (Ag) that is non-ionizing and a silver percentage associated with the mCNPs is about 8% to 15%.

4. The dental resin composition according to claim 1, wherein the predominately 3+ cerium surface charge allows for the hydrogen peroxide reaction to dominate.

5. The dental resin composition according to claim 1, wherein the dental resin comprises a methyl methacrylate polymer resin that includes as polymethyl methacrylate that is cured with a polymethyl methacrylate curing process initiated by tertiary and amine compounds.

6. The dental resin composition according to claim 4, wherein the dental resin includes a methyl methacrylate-based resin that includes monomer polymers and fillers where a poly methyl methacrylate powder incorporates a filler selected from a group consisting of silica, titania and zirconia, and includes benzoyl peroxide as an initiator.

7. The dental resin composition according to claim 1, wherein the dental resin comprises a urethane dimethacrylate-based dental resin that is light curable and includes urethane dimethacrylate (UDMA) combined with ethoxylated bisphenol-A dimethacrylate.

8. The dental resin composition according to claim 1, wherein the bacteria includes at least one of Streptococcus sobrinus and Lactobacilli acidophilus.

9. The dental resin composition according to claim 1, wherein the dental resin comprises a photopolimerisable resin composite that includes urethane dimethacrylate (UDMA) with a filler particle content and a resin matrix of dimethacrylate monomers which are polymerised by free radical reaction initiated by synergy of a photoinitiator system based on camphorquinone and (2,4,6-trimethylbenzoyl)diphenylphosphine oxide.

10. A method of treating teeth, comprising:
forming the resin composition of claim 1 on enamel of each tooth to be treated; and
promoting by the resin composition remineralization of tooth material to further strengthen teeth against decay caused by bacteria in an oral cavity.

11. The method according to claim 10, wherein the forming of the resin composition includes curing the resin composition with an ultraviolet light (UV) source.

12. The method according to claim 10, wherein the predominately 3+ cerium surface charge allows for the hydrogen peroxide reaction to dominate.

13. The method according to claim 10, wherein the metal of the mCNPs is selected from the group consisting of silver, gold, ruthenium, vanadium, copper, titanium, nickel, platinum, titanium, tin, zinc and iron.

14. The method according to claim 10, wherein a metal of the mCNPs is stable metallic silver (Ag) that is non-ionizing and a silver percentage associated with the mCNPs is about 8% to 15%.

15. The method according to claim 10, wherein the forming of the resin composition includes forming a dental resin that includes hot or cold cured methyl methacrylate-based polymer resin.

16. The method according to claim 10, wherein the forming of the resin composition includes forming a dental resin that includes a methyl methacrylate-based resin having PMMA powder, methacrylate monomer polymers and fillers that include titania and silica.

17. The method according to claim 10, wherein the forming the resin composition includes forming a dental resin that includes a urethane dimethacrylate-based dental resin or co-monomers with urethane dimethacrylate-based resins.

18. The method according to claim 10, wherein bacteria includes Streptococcus sobrinus and/or Lactobacilli acidophilus.

19. The method according to claim 10, wherein the forming of the resin composition includes forming a dental resin that includes a photopolimerisable resin composite having urethane dimethacrylate with a filler particle content and a resin matrix of dimethacrylate monomers which are polymerised by free radical reaction initiated by synergy of a photoinitiator system based on camphorquinone and (2,4,6-trimethylbenzoyl)diphenylphosphine oxide.

20. The method according to claim 10, wherein forming the resin composition includes forming a dental resin that includes a liquid component containing methyl methacrylate (MMA) monomer with a crosslinking agent and inhibitor.
